(19) United States
(12) Patent Application Publication
(10) Pub. No.: US 2026/0090699 A1
(43) Pub. Date: Apr. 2, 2026

(54) GAS/WATER VALVE WITH BACKFLOW PREVENTION FOR MEDICAL DEVICES

(71) Applicant: Boston Scientific Scimed, Inc., Maple Grove, MN (US)

(72) Inventors: Kate Beatrice Concannon, Brighton, MA (US); Nathan Thomas Cummings, Worcester, MA (US); Brian Luis, Boylston, MA (US); Jennifer Evelyn Edmondson, North Grafton, MA (US)

(73) Assignee: Boston Scientific Scimed, Inc., Maple Grove, MN (US)

(21) Appl. No.: 19/347,013

(22) Filed: Oct. 1, 2025

Related U.S. Application Data
(60) Provisional application No. 63/702,315, filed on Oct. 2, 2024.

(51) Int. Cl. A61B 1/00

(52) U.S. Cl. CPC A61B 1/00068; A61B 1/0011; A61B 1/00119

(57) ABSTRACT

A gas/water valve for a medical device, including a shaft and a seal assembly. The shaft may include an outer surface, a first opening, a second opening, a third opening located between the first opening and the second opening, and a bore extending from the first opening to the second opening and in fluid communication with the third opening.

The seal assembly may be positioned at the third opening, extend less than an entirety of a circumference around the shaft, and selectively seal the third opening.

### CROSS-REFERENCE TO RELATED APPLICATIONS

This application claims the benefit of U.S. Provisional Patent Application Ser. No. 63/702,315 filed on Oct. 2, 2024, the disclosure of which is incorporated herein by reference.

### FIELD

This disclosure relates generally to medical devices, and particularly to a gas/water valve for use with an endoscope.

### BACKGROUND

Conventionally, endoscope devices have been widely used for performing diagnostic and/or therapeutic treatments. During endoscopic procedures, physicians may use a combination of air, irrigation, and lens wash as a means of flushing debris, cleaning optics, and insufflating the working lumen. A control section of the endoscope may include a suction cylinder and a gas/water cylinder. Valves may be inserted into these cylinders to control various functions of the endoscope. For example, a gas/water valve for an endoscope may be inserted into the gas/water cylinder or channel of the endoscope to provide air and water to the endoscope. When the gas/water valve is in a neutral position, air may escape from a vent in the valve. When insufflation is desired, an operator may place a finger over the vent, which redirects the air towards the distal end of the endoscope thus insufflating the working lumen. When the operator engages the gas/water valve (e.g., by depressing or otherwise actuating the valve), air is redirected to a fluid container and creates pressure in the container that may cause water (or other fluid) to flow towards the distal end of the endoscope.

### SUMMARY

This summary of the disclosure is given to aid understanding, and each of the various aspects and features of the disclosure may advantageously be used separately in some instances, or in combination with other aspects and features of the disclosure in other instances. No limitation as to the scope of the claimed subject matter is intended by either the inclusion or non-inclusion of elements, components, or the like in this summary. Accordingly, while the disclosure is presented in terms of aspects or configurations, it should be appreciated that individual aspects can be claimed separately or in combination with aspects and features of that configuration or any other configuration.

In a first example, a gas/water valve for a medical device may include a shaft having an outer surface, the shaft comprising a first opening, a second opening, a third opening located between the first opening and the second opening, and a bore extending from the first opening to the second opening, the bore being in fluid communication with the third opening, and a seal assembly positioned at the third opening and extending less than an entirety of a circumference around the shaft, the seal assembly configured to selectively seal the third opening.

Alternatively or additionally to any of the examples above, in another example, the gas/water valve may further include a first circumferential seal extending circumferentially around the shaft proximal of the third opening and a second circumferential seal extending circumferentially around the shaft distal of the third opening.

Alternatively or additionally to any of the examples above, in another example, the seal assembly may comprise an extension portion and a seal portion.

Alternatively or additionally to any of the examples above, in another example, the extension portion may be configured to extend into the third opening and engage an interior surface of the shaft and the seal portion may be configured to engage the outer surface of the shaft and seal the third opening.

Alternatively or additionally to any of the examples above, in another example, the seal portion may be configured to translate radially relative to a central axis of the shaft.

Alternatively or additionally to any of the examples above, in another example, the seal portion may be resilient.

Alternatively or additionally to any of the examples above, in another example, the seal portion may be rigid.

Alternatively or additionally to any of the examples above, in another example, the extension portion may be centered on the seal portion.

Alternatively or additionally to any of the examples above, in another example, the gas/water valve may further include an insert positioned in the third opening, the insert comprising one or more securing openings and one or more gas/air openings.

Alternatively or additionally to any of the examples above, in another example, the seal assembly comprises an extension portion configured to extend into a securing opening of the one or more securing openings and a seal portion configured to seal the one or more gas/air openings.

Alternatively or additionally to any of the examples above, in another example, the insert may have a depth proximate the securing opening and the extension portion may have a reduced diameter length substantially equal to the first thickness.

Alternatively or additionally to any of the examples above, in another example, the insert may have a depth proximate the securing opening and the extension portion has a reduced diameter length greater than the depth, such that the extension portion is configured to translate radially relative to the insert as the seal portion adjusts between a closed position and an opened position.

Alternatively or additionally to any of the examples above, in another example, the one or more gas/air openings may comprise a first gas/air opening and a second gas/air opening.

In another example, a gas/water valve assembly for a medical device may include a valve well having a gas outlet, a shaft positioned in the valve well, the shaft having an outer surface, the shaft comprising a first opening, a second opening, a third opening configured to align with the gas outlet, and a bore extending from the first opening to the second opening, the bore being in fluid communication with the third opening, and a seal assembly extending less than an entirety of a circumference around the shaft, the seal assembly configured to selectively block fluid from traveling between the third opening and the gas outlet.

Alternatively or additionally to any of the examples above, in another example, the shaft may have a non-actuated position in the valve well and an actuated position in the valve well and the third opening may be configured to align with the gas outlet when the shaft is in the non-actuated position.

Alternatively or additionally to any of the examples above, in another example, the gas/water valve assembly may further include an insert positioned in the third opening, the insert comprising one or more securing openings and one or more gas/air openings, and wherein the seal assembly may comprise an extension portion configured to extend into a securing opening of the one or more securing openings and a seal portion configured to seal the one or more gas/air openings.

Alternatively or additionally to any of the examples above, in another example, the gas/water valve assembly may further include an insert positioned in the gas outlet, the insert comprising one or more securing openings and one or more gas/air openings, and wherein the seal assembly may include an extension portion configured to extend into a securing opening of the one or more securing openings and a seal portion configured to seal the one or more gas/air openings.

Alternatively or additionally to any of the examples above, in another example, the gas/water valve assembly may further include a wiper seal positioned around the shaft between the first opening and the third opening, and wherein the valve well may comprise a gas inlet configured to align with the first opening and the wiper seal is configured to block fluid from traveling through the gas inlet to the gas outlet between an inner surface of the valve well and the outer surface of the shaft.

In a further example, a method of assembling a gas/water valve may include positioning an insert in a third opening of a shaft comprising an outer surface, a first opening, a second opening, and a bore extending from the first opening to the second opening, wherein the third opening is positioned between the first opening and the second opening and is in fluid communication with the bore and inserting an extension portion of a seal assembly through a securing opening of the insert such that a seal portion of the seal assembly is configured to seal one or more gas/air openings of the insert.

Alternatively or additionally to any of the examples above, in another example, positioning the insert into the third opening of the shaft may comprise over molding the insert at the third opening.

These and other features and advantages of the present disclosure will be readily apparent from the following detailed description, the scope of the claimed invention being set out in the appended claims.

### BRIEF DESCRIPTION OF THE DRAWINGS

The accompanying drawings, which are incorporated in and constitute a part of this specification, illustrate various example configurations and together with the description serve to explain the principles of the present disclosure.

FIG. 1 schematically depicts components of an illustrative configuration of an endoscope;

FIG. 2 schematically depicts components of an illustrative configuration of an endoscope system;

FIG. 3 schematically depicts a perspective view of an illustrative configuration of a gas/water valve;

FIG. 4A schematically depicts a cross-sectional view of the illustrative configuration of the gas/water valve of FIG. 3 in a valve well with the gas/water valve in a neutral position;

FIG. 4B schematically depicts a cross-sectional view of the illustrative configuration of the gas/water valve of FIG. 3 in a valve well with the gas/water valve in a first position;

FIG. 4C schematically depicts a cross-sectional view of the illustrative configuration of the gas/water valve of FIG. 3 in a valve well with the gas/water valve in a second position;

FIG. 5 is a schematic side view of an illustrative insert positioned at an opening in a shaft of a gas/water valve;

FIG. 6 is a schematic side view of an illustrative insert positioned at an opening in a shaft of a gas/water valve;

FIG. 7 is a schematic side view of illustrative sub-openings in a shaft of a gas/water valve;

FIG. 8 is a schematic side view of illustrative sub-openings in a shaft of a gas/water valve;

FIGS. 9A and 9B schematically depict an end view and a cross-section view, respectively, of an illustrative configuration of a seal;

FIG. 10 is a schematic cross-section view of an illustrative configuration of a seal;

FIGS. 11A and 11B depict a schematic cross-section view of a portion of a gas/water valve in a valve well, with a seal of the gas/water valve in a first configuration and a second configuration, respectively;

FIG. 12 depicts a schematic cross-section view of a portion of gas/water valve in a valve well, with a seal of the gas/water valve in an opened configuration;

FIGS. 13A and 13B depict a schematic cross-section view of a portion of gas/water valve in a valve well, with a seal of the gas/water valve in a first configuration and a second configuration, respectively;

FIGS. 14A and 14B depict a schematic cross-section view of a portion of gas/water valve in a valve well, with a seal of the gas/water valve in a first configuration and a second configuration, respectively; and

FIGS. 15A and 15B depict a schematic cross-section view of a portion of gas/water valve in a valve well, with a seal of the valve well in a first configuration and a second configuration, respectively.

While the disclosure is amenable to various modifications and alternative forms, specifics thereof have been shown by way of example in the drawings and will be described in detail. The intention is not to limit the invention to the particular configurations described. On the contrary, the intention is to cover all modifications, equivalents, and alternatives falling within the spirit and scope of the disclosure.

### DETAILED DESCRIPTION

This disclosure is now described with reference to an illustrative medical system that may be used in endoscopic medical procedures. However, it should be noted that reference to this particular procedure is provided only for convenience and not intended to limit the disclosure. A person of ordinary skill in the art would recognize that the concepts underlying the disclosed devices and related methods of use may be utilized in any suitable procedure, medical or otherwise. This disclosure may be understood with reference to the following description and the appended drawings.

The term “distal” refers to a portion farthest away from a user when introducing a device into a patient. By contrast, the term “proximal” refers to a portion closest to the user when placing the device into the patient. As used herein, the terms “comprises,” “comprising,” or any other variation thereof, are intended to cover a non-exclusive inclusion, such that a process, method, article, or apparatus that comprises a list of elements does not necessarily include only those elements, but may include other elements not expressly listed or inherent to such process, method, article, or apparatus. Additionally, terms that indicate the geometric shape of a component/surface refer to exact and approximate shapes.

Configurations of the present disclosure are described with specific reference to a gas/water valve of an endoscope. Such configurations may be used to control the flow of fluid, for a variety of different purposes where it is desired to selectively fluidly couple one or more channels of a device.

Although the present disclosure includes descriptions of a gas/water valve suitable for use with an endoscope system to control insufflation and/or lens wash or irrigation fluid to an endoscope, the devices, systems, and methods herein could be implemented in other medical systems requiring the selective fluid and/or gas coupling of various channels, and for various other purposes.

It is noted that references in the specification to “a configuration”, “some configurations”, “other configurations”, etc., indicate that the configuration(s) described may include a particular feature, structure, or characteristic, but every configuration may not necessarily include the particular feature, structure, or characteristic. Moreover, such phrases are not necessarily referring to the same configuration. Further, when a particular feature, structure, or characteristic is described in connection with a configuration, it would be within the knowledge of one skilled in the art to affect such feature, structure, or characteristic in connection with other configurations, whether or not explicitly described, unless clearly stated to the contrary. That is, the various individual elements described below, even if not explicitly shown in a particular combination, are nevertheless contemplated as being combinable or arrangeable with each other to form other additional configurations or to complement and/or enrich the described configuration(s).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

Conventionally, endoscope devices have been widely used for performing diagnostic and/or therapeutic treatments. During endoscopic procedures, physicians may use a combination of air, irrigation, and lens wash as a means of flushing debris, cleaning optics, and insufflating the working lumen. Gas/water valves may be used for operating gas and/or fluid flow.

Gas/water valves may incorporate a one-way seal to prevent backflow and maintain insufflation pressure in the working lumen. In some cases, one-way seals may partially or fully invert during use. This may increase the amount of force required to depress the valve and/or remove the valve. Additionally, a partially or fully inverted seal may reduce the effectiveness of the seal when insufflation is required. It may be desirable to provide gas/water valves with alternative sealing mechanisms.

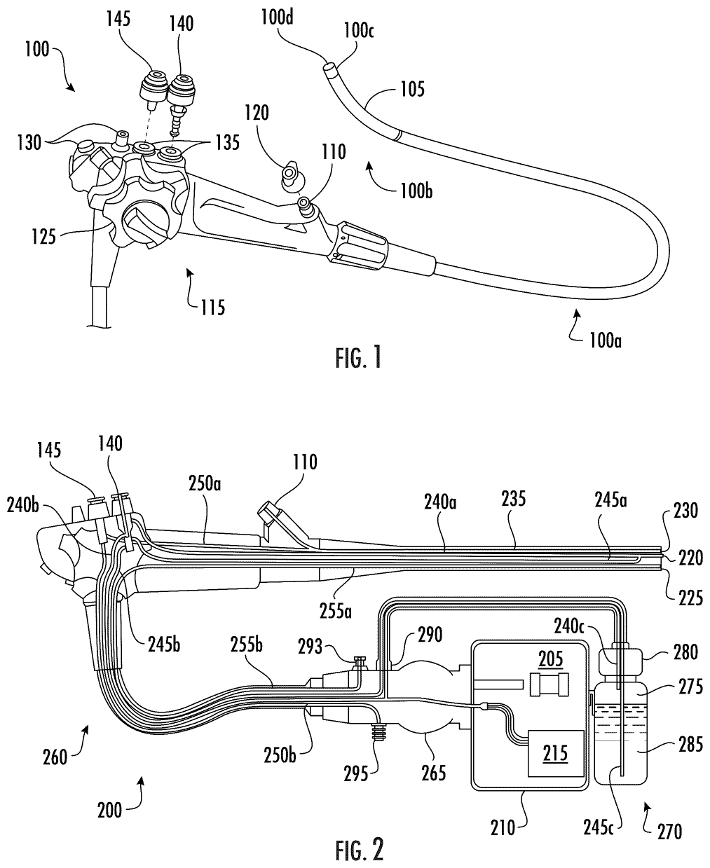

With reference to FIGS. 1 and 2, an illustrative endoscope 100 and endoscope system 200 are depicted that may comprise an endoscope shaft 100a (e.g., an elongate shaft) that is inserted into a patient. A light source 205 feeds illumination light to a distal portion 100b of the endoscope 100, which may house an imager (e.g., CCD or CMOS imager) (not shown). The light source 205 (e.g., lamp) is housed in a video processing unit 210 that processes signals that are input from the imager and outputs processed video signals to a video monitor (not shown) for viewing. The video processing unit 210 also serves as a component of a gas/water feed circuit by housing a pressurizing pump 215, such as an air feed pump, in the unit.

The endoscope shaft 100a may include a distal tip 100c provided at the distal portion 100b of the endoscope shaft 100a and a flexible bending portion 105 proximal to the distal tip 100c. The flexible bending portion 105 may include an articulation joint (not shown) to assist with steering the distal tip 100c. On an end face 100d of the distal tip 100c of the endoscope 100 is a gas/lens wash nozzle 220 for supplying gas to insufflate the interior of the patient at the treatment area and for supplying water to wash a lens covering the imager. An irrigation opening 225 in the end face 100d supplies irrigation fluid to the treatment area of the patient. Illumination windows (not shown) that convey illumination light to the treatment area, and an opening 230 to a working channel 235 extending along the endoscope shaft 100a for passing tools to the treatment area, may also be included on the end face 100d of the distal tip 100c. The working channel 235 extends along the endoscope shaft 100a to a proximal channel opening 110 positioned distal to an operating handle 115 of the endoscope 100. A biopsy valve 120 may be utilized to seal the channel opening 110 against unwanted fluid egress.

The operating handle 115 may be provided with knobs 125 for providing remote 4-way steering of the distal tip via wires connected to the articulation joint in the bendable flexible portion 105 (e.g., one knob controls up-down steering and another knob control for left-right steering). A plurality of video switches 130 for remotely operating the video processing unit 210 may be arranged on a proximal end side of the handle 115. In addition, the handle 115 is provided with dual valve wells 135. One of the valve wells 135 may receive a gas/water valve 140 for operating an insufflating gas and lens water feed operation. A gas supply line 240a and a lens wash supply line 245a run distally from the gas/water valve 140 along the endoscope shaft 100a and converge at the distal tip 100c proximal to the gas/lens wash nozzle 220 (FIG. 2). The other valve well 135 receives a suction valve 145 for operating a suction operation. A suction supply line 250a runs distally from the suction valve 145 along the endoscope shaft 100a to a junction point in fluid communication with the working channel 235 of the endoscope 100.

The operating handle 115 is electrically and fluidly connected to the video processing unit 210, via a flexible umbilical 260 and connector portion 265 extending therebetween. The flexible umbilical 260 has a gas (e.g., air or CO₂) feed line 240b, a lens wash feed line 245b, a suction feed line 250b, an irrigation feed line 255b, a light guide (not shown), and an electrical signal cable (not shown). The connector portion 265 when plugged into the video processing unit 210 connects the light source 205 in the video processing unit with the light guide. The light guide runs along the umbilical 260 and the length of the endoscope shaft 100a to transmit light to the distal tip 100c of the endoscope 100. The connector portion 265 when plugged into the video processing unit 210 also connects the air pump 215 to the gas feed line 240b in the umbilical 260.

A water reservoir or container 270 (e.g., water bottle) is fluidly connected to the endoscope 100 through the connector portion 265 and the umbilical 260. A length of gas supply tubing 240c passes from one end positioned in an air gap 275 between the top 280 (e.g., bottle cap) of the reservoir 270 and the remaining water 285 in the reservoir 270 to a detachable gas/lens wash connection 290 on the outside of the connector portion 265. The detachable gas/lens wash connection 290 may be detachable from the connector portion 265 and/or the gas supply tubing 240c. The gas feed line 240b from the umbilical 260 branches in the connector portion 265 to fluidly communicate with the gas supply tubing 240c at the detachable gas/lens wash connection 290, as well as the air pump 215. A length of lens wash supply tubing 245c, with one end positioned at the bottom of the reservoir 270, passes through the top 280 of the reservoir 270 to the same detachable connection 290 as the gas supply tubing 240c on the connector portion 265. In other configurations, the connections may be separate and/or separated from each other. The connector portion 265 also has a detachable irrigation connection 293 for irrigation supply tubing (not shown) running from a source of irrigation water (not shown) to the irrigation feed line 255b in the umbilical 260. The detachable irrigation connection 293 may be detachable from the connector portion 265 and/or the irrigation supply tubing (not shown). In some configurations, irrigation water is supplied via a pump (e.g., peristaltic pump) from a water source independent (not shown) from the water reservoir 270. In other configurations, the irrigation supply tubing and lens wash supply tubing 245c may source water from the same reservoir. The connector portion 265 may also include a detachable suction connection 295 for suction feed line 250b and suction supply line 250a fluidly connecting a vacuum source (e.g., hospital house suction) (not shown) to the umbilical 260 and endoscope 100. The detachable suction connection 295 may be detachable from the connector portion 265 and/or the suction feed line 250b and/or the vacuum source.

The gas feed line 240b and lens wash feed line 245b are fluidly connected to the valve well 135 for the gas/water valve 140 and configured such that operation of the gas/water valve 140 in the well controls supply of gas or lens wash to the distal tip 100c of the endoscope 100. The suction feed line 250b is fluidly connected to the valve well 135 for the suction valve 145 and configured such that operation of the suction valve in the well controls suction applied to the working channel 235 of the endoscope 100.

Referring to FIG. 2, an illustrative operation of an endoscope system 200, including an endoscope such as endoscope 100 above, is explained. Air from the air pump 215 in the video processing unit 210 is flowed through the connector portion 265 and branched to the gas/water valve 140 on the operating handle 115 through the gas feed line 240b in the umbilical 260, as well as through the gas supply tubing 240c to the water reservoir 270 via the connection 290 on the connector portion 265. When the gas/water valve 140 is in a neutral position, without the user's finger on the valve, air is allowed to flow out of the valve to atmosphere. In a first position, the user's finger is used to block the vent to atmosphere. Gas is allowed to flow from the gas/water valve 140 down the gas supply line 240a and out the distal tip 100c of the endoscope 100 in order to, for example, insufflate the treatment area of the patient. When the gas/water valve 140 is pressed downward to a second position, gas is blocked from exiting the valve, allowing pressure of the air passing from the air pump 215 to rise in the water reservoir 270. Pressurizing the water source forces water out of the lens wash supply tubing 245c, through the connector portion 265, umbilical 260, through the gas/water valve 140 and down the lens wash supply line 245a, converging with the gas supply line 240a prior to exiting the distal tip 100c of the endoscope 100 via the gas/lens wash nozzle 220. Air pump pressure may be calibrated to provide lens wash water at a relatively low flow rate compared to the supply of irrigation water.

The volume of the flow rate of the lens wash is governed by gas pressure in the water reservoir 270. When gas pressure begins to drop in the water reservoir 270, as water is pushed out of the reservoir 270 through the lens wash supply tubing 245c, the air pump 215 replaces lost air supply in the reservoir 270 to maintain a substantially constant pressure, which in turn provides for a substantially constant lens wash flow rate. In some configurations, a filter (not shown) may be placed in the path of the gas supply tubing 240c to filter-out undesired contaminants or particulates from passing into the water reservoir 270. In some configurations, outflow check valves or other one-way valve configurations (not shown) may be placed in the path of the lens wash supply tubing to help prevent water from back-flowing into the reservoir 270 after the water has passed the valve.

A relatively higher flow rate of irrigation water is typically required compared to lens wash, since a primary use is to clear the treatment area in the patient of debris that obstructs the user's field of view. Irrigation is typically achieved with the use of a pump (e.g., peristaltic pump), as described. In configurations with an independent water source for irrigation, tubing placed in the bottom of a water source is passed through the top of the water source and threaded through the head on the upstream side of the pump.

Tubing on the downstream side of the pump is connected to the irrigation feed line 255b in the umbilical 260 and the irrigation supply line 255a endoscope 100 via the irrigation connection 293 on the connector portion 265. When irrigation water is required, fluid is pumped from the water source by operating the irrigation pump, such as by depressing a footswitch (not shown), and flows through the irrigation connection 293, through the irrigation feed line 255b in the umbilical, and down the irrigation supply line in the endoscope shaft 100a of the endoscope to the distal tip 100c. In order to equalize the pressure in the water source as water is pumped out of the irrigation supply tubing, an air vent (not shown) may be included in the top 280 of the water reservoir 270. The vent allows atmospheric air into the water source preventing negative pressure build-up in the water source, which could create a vacuum that suctions undesired matter from the patient back through the endoscope toward the water source. In some configurations, outflow check valves or other one-way valve configurations (not shown), similar to the lens wash supply tubing 245c, may be placed in the path of the irrigation supply tubing to help prevent back-flow into the reservoir after water has passed the valve. In some cases, irrigation water may be supplied from the water reservoir 270. Some illustrative systems where the supply tubing for irrigation and lens wash are connected to and drawn from a single water reservoir are described in commonly assigned U.S. patent application No. Ser. No. 17/558,239, titled INTEGRATED CONTAINER AND TUBE SET FOR FLUID DELIVERY WITH AN ENDOSCOPE and U.S. patent application Ser. No. 17/558,256, titled TUBING ASSEMBLIES AND METHODS FOR FLUID DELIVERY, the disclosures of which are hereby incorporated by reference.

When the suction valve 145 is in a neutral position or rest state, without the user's finger on and/or depressing the valve, air is allowed to flow into and/or out of the suction valve 145 to atmosphere. When suction is desired, the user's finger is used to block a vent to atmosphere as well as to depress the suction valve. Depressing the suction valve 145 may move the suction valve 145 within the valve well 135 to fluidly couple the working channel 235 with the suction pump (not explicitly shown) via the suction supply line 250a.

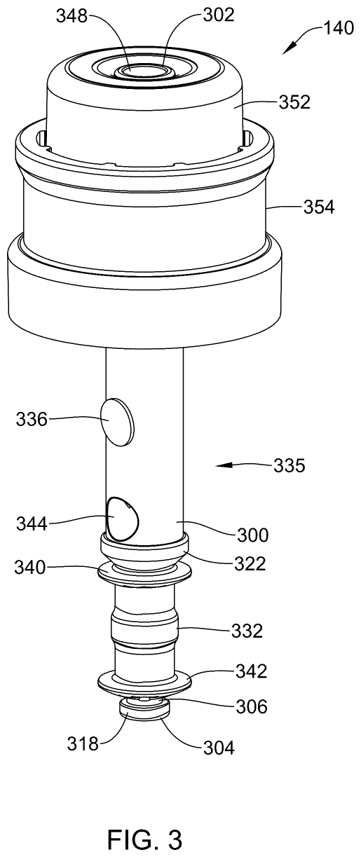

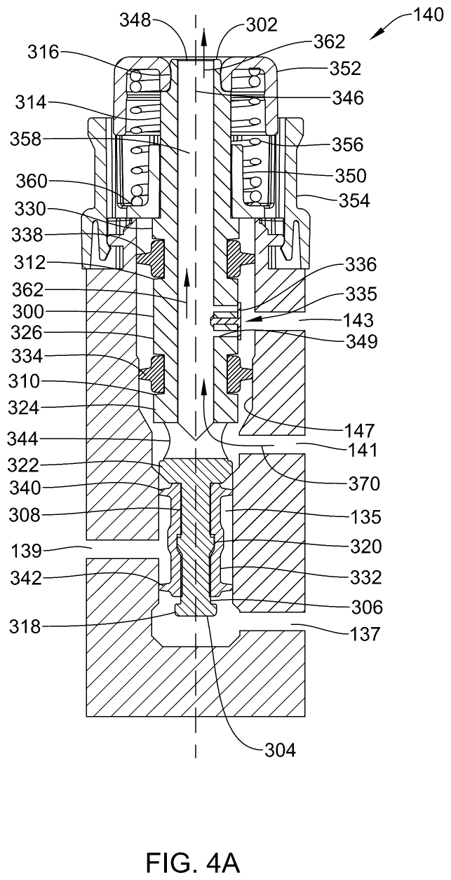

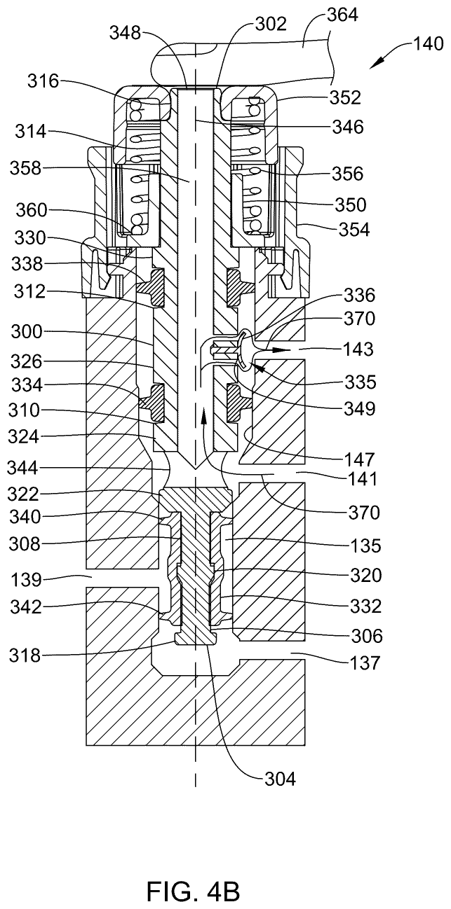

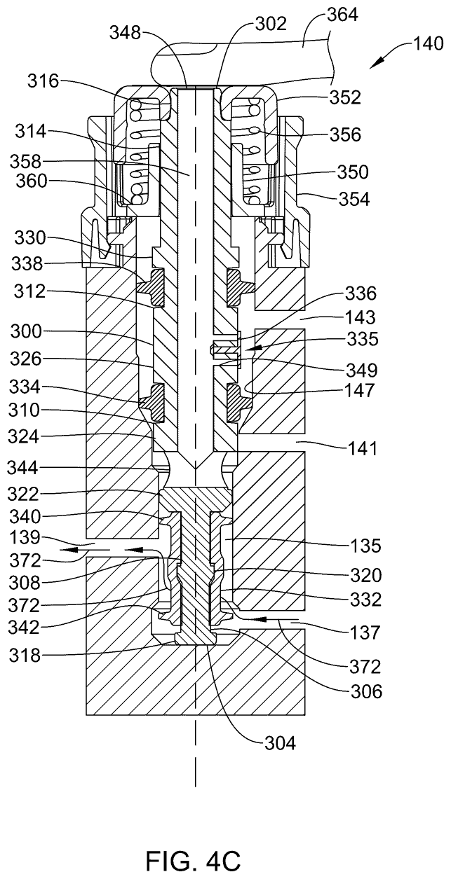

FIG. 3 is a schematic perspective view of an illustrative configuration of the gas/water valve 140 that may be used with the endoscope 100. FIG. 4A is a schematic cross-section view of the configuration of the gas/water valve 140 depicted in FIG. 3 disposed within the valve well 135 with the gas/water valve 140 in a neutral position (e.g., a non-actuated position). FIG. 4B is a schematic cross-section view of the configuration of the gas/water valve 140 depicted in FIG. 3 disposed within a valve well 135 with the gas/water valve 140 in a first position (e.g., a non-actuated position). FIG. 4C is a schematic cross-section view of the configuration of the gas/water valve 140 depicted in FIG. 3 disposed within a valve well 135 with the gas/water valve 140 in a second position (e.g., an actuated position).

Referring to FIGS. 3 and 4A-4C, the gas/water valve 140 may have a stem or shaft 300 extending from a first or proximal end 302 to a second or distal end 304. The shaft 300 may include a plurality of grooves 306, 308, 310, 312, 314, 316 and a plurality of ridges 318, 320, 322, 326, 330 that are molded and/or machined as part of the shaft 300. Said differently, the outer diameter or cross-sectional dimension of the shaft 300 may intermittently increase and/or decrease along the length thereof from the proximal end 302 to the distal end 304, where the outer diameter or cross-sectional dimension of the shaft 300 is defined by an outer surface of the shaft 300. In some cases, the changes in cross-sectional dimension may occur in an abrupt or stair-step manner. In other examples, the changes in the cross-sectional dimension may be gradual, tapered, sloped, or non-existent. The gas/water valve 140 may further include a plurality of seals 332, 334, 336, 338 positioned along a length of the shaft 300. In addition, gas/water valve 140 may also include an inner ring 350, a button cap 352, an outer ring 354, and a biasing or resilient member 356, such as, but not limited to, a spring, rubber, elastic, or the like.

Generally, the seals 332, 334, 336, 338 may be configured to selectively fluidly isolate or fluidly couple portions of the gas/water valve 140 and/or the valve well 135. For example, depending on the position of the gas/water valve 140 relative to the valve well 135 and/or a position of the user's finger 364, the seals 332, 334, 336, 338 may be positioned to selectively fluidly couple fluid paths of the endoscope 100 and endoscope system 200. As will be described in more detail herein, the seals 332, 334, 336, 338 may prevent gas/air from entering the water channel (e.g., water inlet 137 and/or water outlet 139) and prevent gas/air from exiting anywhere other than outside of a vent hole or opening 348 on the top of the shaft 300 or through a gas/air outlet 143 in the valve well 135.

In some configurations, one or more of the plurality of seals 332, 334, 336, 338 can be fixedly or permanently attached to the shaft 300, such as, for example, by over-molding. In other configurations, one or more of the plurality of seals 332, 334, 336, 338 can be removably attached to or coupled to the shaft 300, such as for example, by sliding the seal onto the shaft 300.

The seals 332, 334, 336, 338 may be formed from any material suitable for sealing a portion of the shaft 300. For example, each or one or more of the plurality of seals 332, 334, 336, 338 can be formed from polyurethane, polyurea, polyether(amide), polyether block amide (PEBA), thermoplastic elastomeric olefin, copolyester, and styrenic thermoplastic elastomer, carbon fiber, glass fiber, ceramics, methacrylates, poly (N-isopropylacrylamide), poly(ethylene oxide)-poly(propylene oxide)-poly(ethylene oxide) (PEO-PPO-PEO) co-polymers, rubber, plastic (e.g., polycarbonates), acrylonitrile butadiene styrene (ABS), methylmethacrylate acrylonitrile butadiene styrene (MABS), silicone, etc., or combinations thereof.

A first seal 332 may be positioned in a first groove 306 and may include a first sealing region 340 positioned proximal to a water outlet 139 of the valve well 135 and a second sealing region 342 positioned distal to the water outlet 139. The first and second sealing regions 340, 342 may contact a surface of the valve well 135 (e.g., an inner surface of the valve well 135) to provide a seal between the gas/water valve 140 and the valve well 135. In some examples, the first sealing region 340 and/or the second sealing region 342 may each be or form wiper seals.

In the neutral or first position (FIGS. 4A and 4B, respectively), the second sealing region 342 may be positioned proximal to a water inlet 137 of the valve well 135 and while in the second position (FIG. 4C), the shaft 300 of the gas/water valve 140 may be distally displaced to position the second sealing region 342 distal to or generally aligned with the water inlet 137. When the gas/water valve 140 is in the neutral position or the first position, the second sealing region 342 fluidly isolates the water outlet 139 from the water inlet 137. Further, in the neutral position, the first position, or the second position, the first sealing region 340 fluidly isolates the water inlet 137 and the water outlet 139 from a gas/air inlet 141 and a gas/air outlet 143. In some cases, a diameter of the valve well 135 may increase proximate the water inlet 137 to allow the water inlet 137 to be fluidly coupled with the cavity of the valve well 135 and the water outlet 139 without requiring part or all of the second sealing region 342 to be positioned distal to the water inlet 137.

A second seal 334 (e.g., a circumferential seal) may be positioned within another groove 310 proximal to the gas/air inlet 141 and distal of a third opening 349 (example first and second opening are discussed below). The second seal 334 may contact the inner surface of the valve well 135 to provide a seal to fluidly isolate the gas/air inlet 141 from the gas/air outlet 143 along a path between an outer surface of the stem or shaft 300 and an inner surface 147 of the valve well 135. In some examples, the second seal 334 may be a wiper seal, but other suitable types of seals are contemplated.

A third seal 336 (e.g., an intermediate or side sealing member) may form an entirety of or at least a portion of a seal assembly 335. The seal assembly 335 may be positioned at, proximate to, and/or within the third opening 349 in the stem or shaft 300 proximal of the second seal 334. The third seal 336 may be configured to selectively seal the third opening 349 and move or adjust between an opened to allow gas out of the third opening 349 and a closed position to prevent gas from passing through the third opening 349. The seal assembly 335 and/or the third seal 336 may extend an entirety around a circumference of the shaft 300 or less than an entirety around the circumference of the shaft 300 (e.g., as depicted in FIGS. 3-4C). For example, the third seal 336 may be located at or proximate the third opening 349. In some examples, the third seal 336 may include at a first portion fixed relative to the third opening 349 and a second portion configured to adjust relative to the third opening 349, but other suitable configurations are contemplated. The third seal 336 may be secured to and/or relative to the stem or shaft 300 using any suitable fixation techniques including, but not limited to, adhesives, plastic welding, thermal bonding, friction fits, inserts, and/or the like. As will be described in more detail herein, the third seal 336 may be configured to selectively fluidly couple the gas/air inlet 141 of the valve well 135 with the gas/air outlet 143 of the valve well 135.

A fourth seal 338 (e.g., a circumferential seal) may be positioned within another groove 312 proximal of the gas/air inlet and outlet 141, 143 and proximal of the third opening 349. The fourth seal 338 may contact the inner surface 147 of the valve well 135 to provide a seal to fluidly isolate the gas/air inlet and outlet 141, 143 from atmosphere external to the gas/water valve 140.

A first opening 344 of the shaft 300 may be fluidly coupled with a bore or lumen 358 extending proximally from the first opening 344 to the vent opening 348 (e.g., a second opening). The third opening 349 (e.g., an intermediate opening) may be located between the first opening and the second opening and/or between the second seal 334 and the fourth seal 338, and may be in fluid communication with the bore 358. When the gas/water valve 140 is in the neutral, resting, or first position, the third opening 349 may be aligned with the gas/air outlet 143. In some configurations, the first opening 344 may extend from a first side to a second opposing side of the shaft 300. Said differently, the first opening 344 may extend through a thickness of the shaft 300. The third opening 349 may extend from a single side of the shaft 300 and/or may extend from one or more additional sides of the shaft 300, which may or may not be opposite of one other side of the shaft 300 through which the third opening extends. In some examples, the shaft 300 and/or other suitable portions of the gas/water valve 140 and the valve well 135 may be keyed with respect to one another to facilitate aligning the first opening 344 with the gas/air inlet 141 and/or aligning the third opening 349 with the gas/air outlet 143, but other suitable configurations without keyed features are contemplated. Although the shaft 300 is depicted as having a same or similar outer diameter at the first opening 344 and the third opening 349, the shaft 300 may have a greater outer diameter at or proximate the third opening 349 than at or proximate the first opening 344 or at or proximate the first opening 344 than at or proximate the third opening 349.

When the gas/water valve 140 is in a resting position and the vent opening 348 is not covered by an operator, gas/air may enter the gas/water valve 140 via the first opening 344, travel through the bore 358, and exit the gas/water valve 140 via the vent opening 348, as shown at arrows 362 in FIG. 4A. When gas/water valve 140 is in a first configuration and the vent opening 348 is covered by the finger 364 of the operator, gas/air may enter the gas/water valve 140 via the first opening 344, travel through the bore 358, and exit the gas/water valve 140 via the third opening 349, as shown at arrows 370 in FIG. 4B to provide insufflation.

The bore 358 may extend colinearly with a longitudinal axis 346 of the gas/water valve 140 and/or the first and third openings 344, 349 may extend generally orthogonal to the longitudinal axis 346 of the gas/water valve 140. Alternatively or additionally, one or both of the first and third openings 344, 349 may extend at non-orthogonal angles relative to the longitudinal axis 346 of the gas/water valve 140, as desired. Other suitable configurations are contemplated.

The inner ring 350 may have a diaphragm or collar 360 extending from an outer circumference of the inner ring 350 towards the internal circumference of the outer ring 354. The resilient member 356 may be installed to be positioned between the inner ring 350 and the button cap 352 such that one end of the resilient member 356 is secured to the diaphragm or collar 360 and the other end to the button cap 352. In some configurations, the inner ring 350 is a single monolithic internally molded part of the outer ring 354 while in other configurations these may be two separate components. In various configurations, the outer ring 354 (along with the inner ring 350 when molded as a monolithic part of the outer ring 354) is molded using material of sufficient rigidity. The inner ring 350, outer ring 354, and the button cap 352 respectively define internal bores for receiving the proximal end 302 or vent opening 348 of the shaft 300. The proximal end 302 of the shaft 300 may be placed through the inner ring 350 and the resilient member 356 and secured to the button cap 352. When assembled, the diaphragm or collar 360 of the inner ring 350 may rest upon a ridge 330.

Referring to FIG. 4A, in the neutral or resting position (e.g., a neutral or resting configuration), the vent opening 348 is uncovered or unblocked. Gas/air may enter the valve well 135 via the gas/air inlet 141. Gas enters the gas/water valve 140 via the opening 344, travels through the bore 358, and exits the gas/water valve 140 via the vent opening 348, as shown at arrows 362. The second seal 334 provides a gas-tight seal between the outer surface of the shaft 300 of the gas/water valve 140 and the inner surface 147 of the valve well 135 between the gas/air inlet 141 and the gas/air outlet 143 to prevent gas/air from entering the gas/air outlet 143 when the gas/water valve 140 is in the neutral position. The third seal 336 provides a gas-tight seal between the third opening 349 and the gas/air outlet 143 to prevent gas/air from entering the gas/air outlet 143 when the gas/water valve 140 is in the neutral position.

Referring to FIG. 4B, in the first position, the user covers or blocks the vent opening 348, using, for example, but not limited to, a finger 364. When the vent opening 348 is blocked, gas/air that flows into the valve well 135 and the bore 358 via the gas/air inlet 141 and the first opening 344 is prevented from exiting the gas/water valve 140 via the vent opening 348. The gas/air accumulates in the valve well 135 and the bore 358 of the shaft 300 and the pressure within the bore 358 increases. The pressure generated by the accumulating gas/air in the bore 358 may pass through the third opening 349 and push against a proximal or inner surface of the third seal 336. At least a portion of the third seal 336 may be movably secured relative to the shaft 300 and/or the third opening 349 such that the pressure or air flow through the third opening 349 displaces an entirety of or at least a portion of the third seal 336 such that the gas/air in the bore 358 pass through the third opening 349 across the third seal 336 and out of the gas/air outlet 143 to provide insufflation, as shown at arrows 370. The third seal 336 may remain at or in an opened position or configuration relative to the shaft 300 and/or the third opening 349 until a pressure on a first side of the third seal 336 (e.g., in the bore 358) at or a flow rate of gas/air through the third opening 349 is insufficient to maintain the third seal 336 in the opened position.

Referring now to FIG. 4C, when the gas/water valve 140 is in the second position, the gas/water valve 140 is axially displaced in the distal direction while the vent opening 348 remains blocked or covered. In the second position, the first seal 332 is axially displaced in the distal direction to fluidly couple the water inlet 137 and the water outlet 139. Water flows into the valve well 135 via the water inlet 137, fills the annular cavity surrounding the first seal 332, and exits the valve well 135 via the water outlet 139, as shown at arrows 372. As the gas/water valve 140 is distally displaced, a portion of the second seal 334 engages the inner surface 147 of the valve well 135 proximate a distal edge of the gas/air inlet 141 and a portion of the second seal 334 may engage the inner surface 147 of the valve well 135 to prevent gas/air from entering the bore 358 and/or the gas/air outlet 143. Alternatively or additionally, as the gas/water valve 140 is displaced distally, an outer surface of the shaft 300 and/or a further seal may be utilized to engage the inner surface 147 of the valve well 135 proximate a distal edge of the gas/air inlet 141 and the second seal 334 may engage the inner surface 147 of the valve well 135 to prevent gas/air from entering the bore 358 and/or the gas/air outlet 143. This may cause pressurization of the water container 270 to provide pressurized water to the distal end of the endoscope via the water inlet 137 and the water outlet 139.

To facilitate positioning the third seal 336 at the third opening 349, one or more sub-openings 374 may be located at or proximate the third opening 349. At least one of the sub-openings 374may be configured to engage and/or secure the third seal in, at, and/or proximate the third opening. At least one of the sub-openings 374 may be configured to allow gas/air form the bore 358 to pass there through when the third seal 336 is in the opened position. In some examples, the sub-openings 374 may be formed in the shaft 300 in, at, and/or proximate the third opening 349 and/or the sub-openings 374 may be formed in an insert positioned in, at, and/or proximate the third opening 349.

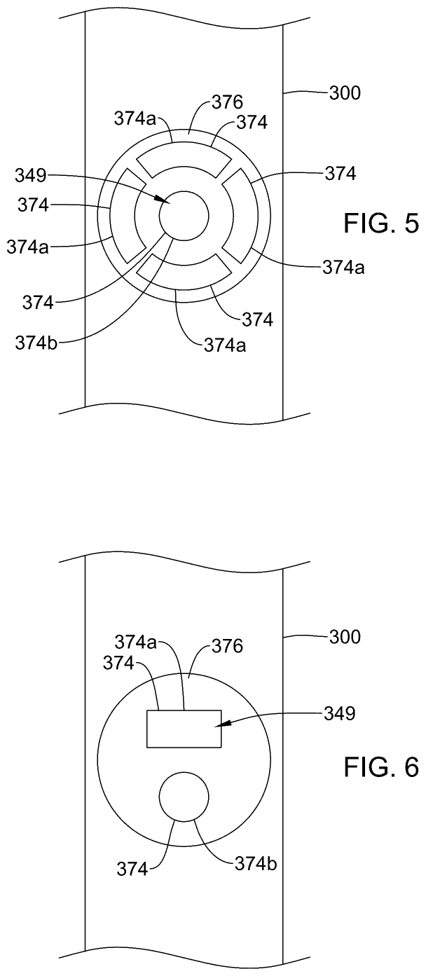

The sub-openings 374 may have any suitable configuration. Any suitable number of sub-openings 374 may be utilized including, but not limited to, a single sub-opening 374, two sub-openings 374, three sub-openings 374, four sub-openings 374, five sub-openings 374, and/or other suitable number of sub-openings 374. The sub-openings 374 may have any suitable arrangement with respect to one another including, but not limited to, a concentric arrangement, a circular arrangement, circumferentially-spaced arrangement, a stacked arrangement, a symmetrical arrangement, an asymmetrical arrangement, and/or other suitable arrangement. The sub-openings 374 may have any suitable cross-sectional shapes including, but not limited to, circular shapes, rectangular shapes, square shapes, arc-shapes, tapered shapes. The sub-openings 374 may have any suitable shape through a wall in which the sub-opening 374 are formed which may or may not be designed to achieve a certain flow rate of fluid and/or a certain direction of fluid passing through the sub-openings 374. In one example, the shape of the sub-openings 374 through the wall in which the sub-openings are formed may be configured to direct the flow of fluid toward the gas/air outlet 143. FIGS. 5-8 depict illustrative configurations of sub-openings 374, but other suitable configurations of the sub-openings 374 are contemplated in addition to or as alternatives to the configurations depicted in FIGS. 5-8.

FIG. 5 depicts a schematic side of view a portion of an illustrative configuration of the shaft 300 at the third opening 349, where an insert 376 is positioned at the third opening 349. The insert 376, when included, may be part of the seal assembly 335. The insert 376 may have any suitable configuration. In some examples, the insert 376 may be shaped and/or sized to fit within the third opening 349 and have an exterior surface flush with the exterior surface of the shaft 300. Additionally and/or alternatively, at least a portion of the insert 376 may be sized and/or shaped to have an exterior surface inset in the third opening 349 and/or offset to an exterior of the exterior surface of the shaft 300. Although the insert 376 is depicted in FIG. 5 as being circular, the outer circumference of the insert 376 may have any other suitable shape that may or may not be dictated by an inner surface of the third opening 349.

The insert 376 may be formed from any suitable material. Example suitable materials for forming the insert 376 include, but are not limited to, flexible materials, rigid materials, resilient materials, metals, polymers, polyurethane, polyurea, polyether(amide), polyether block amide (PEBA), thermoplastic polymers, thermoplastic polyurethane (TPU), thermoplastic elastomer (TPE) thermoplastic elastomeric olefin, copolyester, styrenic thermoplastic elastomer, carbon fiber, glass fiber, ceramics, methacrylates, poly (N-isopropylacrylamide), poly(ethylene oxide)-poly(propylene oxide)-poly(ethylene oxide) (PEO-PPO-PEO) co-polymers, rubber, plastic (e.g., polycarbonates), acrylonitrile butadiene styrene (ABS), methylmethacrylate acrylonitrile butadiene styrene (MABS), silicone, etc., or combinations thereof.

The insert 376 may be formed in any suitable manner. For example, the insert 376 may be formed from a stamping procedure, an injection molding procedure, an over-molding procedure, and/or other suitable procedures. In one example, the insert 376 may be injection molded separate from the shaft 300 and inserted into and secured within the third opening 349. The insert 376 may be secured within the third opening 349 in any suitable manner including, but not limited to, via adhesive, a friction fit, a threaded fit, a snap fit, etc. or combinations thereof. In another example, the insert 376 may be over-molded onto and/or into the third opening 349 of the shaft 300.

The insert 376 may include one or more of the sub-openings 374. Although all of the sub-openings 374 are depicted in FIG. 5 as being on or in the insert 376, one or more sub-openings 374 may be located on or in the shaft 300 in addition to and/or as an alternative to the sub-openings 374 on or in the insert 376. In some example configurations, the insert 376 may include one or more gas/air sub-openings 374a (e.g., a gas/air opening or first sub-openings) configured to allow gas/air to pass therethrough and/or one or more securing sub-openings 374b (e.g., a securing opening or a second sub-openings) configured to receive and/or engage a securing portion of the third seal 336.

Any suitable number of gas/air sub-openings 374a may be utilized and/or any suitable number of securing sub-openings 374b may be utilized. In one example, the insert 376 may include a single securing sub-opening 374b centered on the insert 376 and four gas/air sub-openings 374a circumferentially spaced from one another and concentrically positioned about the single securing sub-opening 374b, as depicted in FIG. 5. The four gas/air sub-openings 374a may be shaped as four-side arcs and the securing sub-opening 374b may have a circular shape, but other suitable shapes are contemplated. Although the securing sub-opening 374b is depicted as being circular and only one securing sub-opening 374b is depicted, one or more securing sub-openings 374b having any suitable shape may be utilized. Other suitable configurations of the insert 376 and/or the sub-openings 374 are contemplated.

FIG. 6 depicts a schematic side of view of a portion of an illustrative configuration of the shaft 300 at the third opening 349, where the insert 376 including a plurality of sub-openings 374 is positioned at the third opening 349. Although other configurations are contemplated, an exterior surface of the insert 376 may be flush with an exterior surface of the shaft 300. In the example configuration of the insert 376 depicted in FIG. 6, a single gas/air sub-opening 374a and a single securing sub-opening 374b are vertically spaced from one another, with the gas/air sub-opening 374a located proximal of the securing sub-opening 374b. The single gas/air sub-openings 374a may have a rectangular shape and the securing sub-opening 374b may have a circular shape, but other suitable shapes are contemplated.

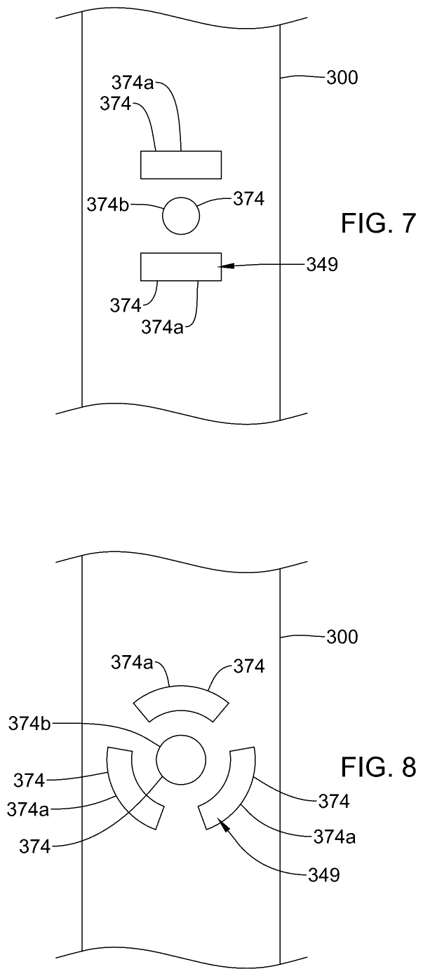

FIG. 7 depicts a schematic side of view of a portion of an illustrative configuration of the shaft 300 at the third opening 349, where a plurality of sub-openings 374 are positioned at the third opening 349. An outer-most circumference of the sub-openings 374 may be flush with an exterior surface of the shaft 300, as depicted for example in FIG. 7, but it is contemplated that at least a portion of the outer-most circumference of the sub-openings 374 formed in the shaft 300 may be inset from and/or offset outward from the exterior surface of the shaft 300. In the example configuration of the sub-openings 374 depicted in FIG. 7, two gas/air sub-opening 374a and a single securing sub-opening 374b are vertically spaced from one another, with securing sub-opening 374b centrally spaced between the two gas/air sub-openings 374a located proximal of and distal of the securing sub-opening 374b. The two gas/air sub-openings 374a may have rectangular arc shapes (e.g., four-sided arcs) and the securing sub-opening 374b may have a circular shape, but other suitable shapes are contemplated.

The sub-openings 374 may be formed in the shaft 300 in any suitable manner. For example, the sub-openings 374 may be formed in a wall of the shaft 300 so as to define at least part of the third opening 349 using any suitable technique including, but not limited to, molding, punching, boring, etching, and/or other suitable manufacturing techniques.

FIG. 8 depicts a schematic side of view of a portion of an illustrative configuration of the shaft 300 at the third opening 349, where a plurality of sub-openings 374 are positioned at the third opening 349. In the example configuration of the sub-openings 374 depicted in FIG. 8, three gas/air sub-opening 374a may be circumferentially spaced from one another and concentric about a single securing sub-opening 374b. The three gas/air sub-openings 374a may have rectangular arc shapes and the securing sub-opening 374b may have a circular shape, but other suitable shapes are contemplated.

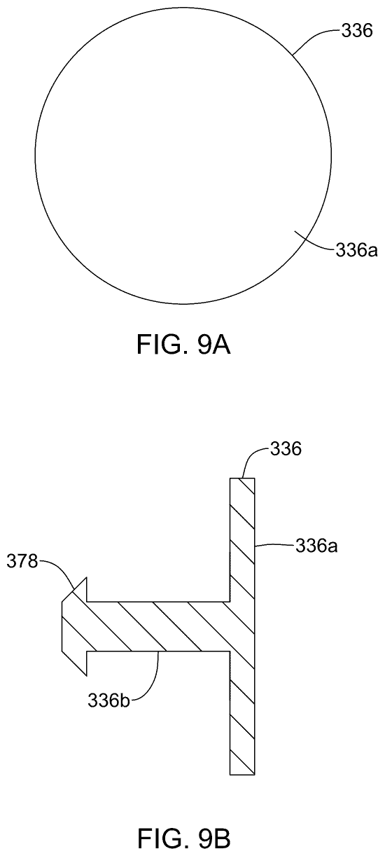

The third seal 336 may have any suitable configuration configured to block fluid flow between the third opening 349 and the gas/air outlet 143 when in a closed position and allow fluid flow between the third opening 349 and the gas/air outlet 143 when in the opened position. In some examples, the third seal 336 may include one or more seal portions 336a (e.g., a first portion) configured to selectively block fluid flow between the third opening 349 and the gas/air outlet 143 (e.g., via the sub-openings 374) and one or more extension portions 336b configured to maintain the third seal 336 in, at, and/or proximate the third opening 349. In some examples, the seal portion 336a may overlap the sub-openings 374 when in the closed position and the extension portion 336b may be configured to extend into the third opening 349 and engage an interior surface of the shaft 300. FIGS. 9A and 9B depict a schematic end view and a schematic cross-section view, respectively, of an illustrative configuration of the third seal 336.

The third seal 336 may be formed from any suitable material including, but not limited to, metals, polymers, rigid materials, flexible materials, resilient materials, and/or other suitable materials. In some examples, the third seal 336 may be entirely formed from a flexible material. In some examples, the third seal 336 may be entirely formed from a rigid material. In some examples, the third seal 336 may be formed entirely from a resilient material. In some examples, the third seal 336 may be formed from two or more of a rigid material, a flexible material, a resilient material, and an elastomeric material. In one example, the seal portion 336a of the third seal 336 may be formed from a flexible material and the extension portion 336b may be formed from a rigid material. In another example, the seal portion 336a and the extension portion 336b may be formed from one or more elastomeric materials such that the extension portion 336b may be passed through and engage the securing sub-opening(s) 374b and the seal portion 336a may be flexible or resilient so as to be configured to adjust between the opened position and the closed position. Other suitable material configurations of the third seal 336 are contemplated for forming resilient, flexible, and/or rigid third seals 336 or forming portions thereof.

The third seal 336 may be formed in any suitable manners. For example, the third seal 336 may be formed using a molding technique, an over-molding technique, an injection molding technique, a lathe technique, and/or other suitable techniques.

The seal portion 336a may have any suitable configuration. For example, the seal portion 336a may have a circular circumference, a rectangular circumference, an arc circumference, a triangular circumference, and/or other suitable circumference. In one example and as depicted in FIG. 9A, the seal portion 336a may have a circular circumference, but other suitable configurations are contemplated.

The seal portion 336a of the third seal 336 may have any suitable depth. For example, and as depicted in FIG. 9B, the seal portion 336a may have a constant depth. In some examples, one or more portions of the seal portion 336a may have a different depth than a depth of one or more other portions of the seal portion 336a. In one example, the depth of the seal portion 336a may taper radially outward (e.g., become thinner in the radially outward direction relative to a central axis or location of the seal portion 336a) for at a least a portion of a circumference of the seal portion 336a. Although other suitable depths are contemplated, the depth of the seal portion 336a may be 2 millimeters (mm) or less, 1 mm or less, and/or other suitable depths. In one example, the depth of the seal portion 336a may be 1 mm, but other suitable configurations are contemplated.

The extension portion 336b of the third seal 336 may have any suitable configuration. In some examples, the extension portion 336b may be an elongate component with one end at the seal portion 336a and a second end having a fixation component 378, as depicted for example in FIG. 9B. The portion of the extension portion 336b between the seal portion 336a and the fixation component 378 may be a reduced diameter portion having a diameter or width that is less than a diameter or width of the fixation component 378. The extension portion 336b may be centrally located on the seal portion 336a, but other suitable configurations are contemplated.

The fixation component 378 may be configured to engage a surface as the seal portion 336a adjusts between the closed and opened positions to maintain the third seal 336 at a location aligned with the third opening 349 and/or the gas/air outlet 143. In one example, the fixation component 378 may have a tapered portion at least partially wider than or a larger diameter than the extension portion 336b between the seal portion 336a and the fixation component 378 (e.g., the reduced diameter portion) configured to facilitate inserting the extension portion 336b into an opening and preventing or mitigating unintentional withdrawal of the extension portion 336b from the opening, as depicted for example in FIG. 9B. An intentional force may be applied to the third seal 336 to remove the seal 336 from a location proximate the third opening 349 after the third seal 366 has been secured at the location. Other suitable configurations of the fixation component 378 are contemplated.

The extension portion 336b of the third seal 336 between the seal portion 336a and the fixation component 378 may have any suitable length. In some examples, the length of the extension portion 336b between the seal portion 336a and the fixation component 378 (e.g., a reduced diameter length) may be substantially equal to a length or depth of the securing sub-opening 374b to fix the extension portion 336b of the third seal 336 relative to the securing sub-opening 374b. In some examples, the length of the extension portion 336b between the seal portion 336a and the fixation component 378 (e.g., the reduced diameter length) may be longer than a length or depth of the securing sub-opening 374b to allow the extension portion 336b of the third seal 336 to translate and/or otherwise adjust relative to the securing sub-opening 374b as the seal portion 336a adjusts between the closed position and the opened position.

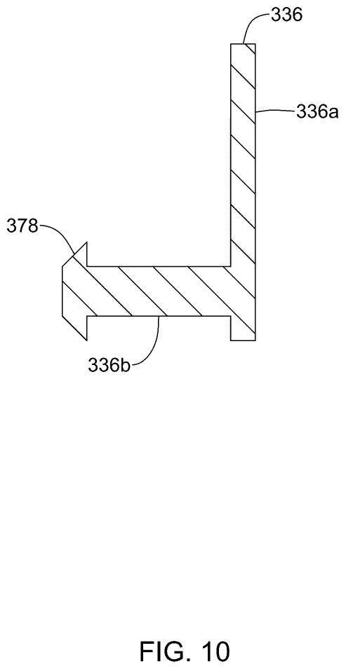

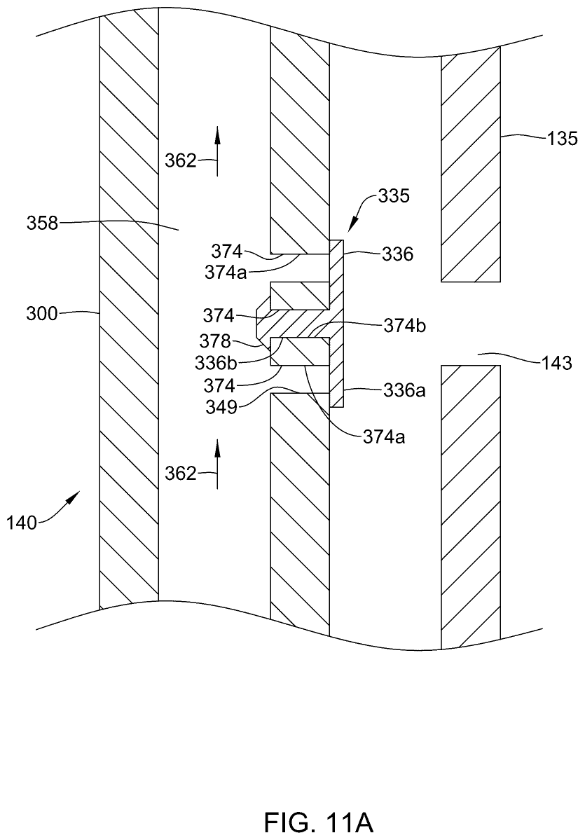

FIG. 10 depicts a cross-sectional view of an illustrative configuration of the third seal 336. As depicted in FIG. 10, the extension portion 336b may be offset in a distal direction from a center of the seal portion 336a of the third seal 336. Other suitable configurations in which the extension portion 336b is offset from a center of the seal portion 336a in one or more other manners are contemplated and/or in which a plurality of extension portions 336b extending from or are otherwise in communication with the seal portion 336a are contemplated.

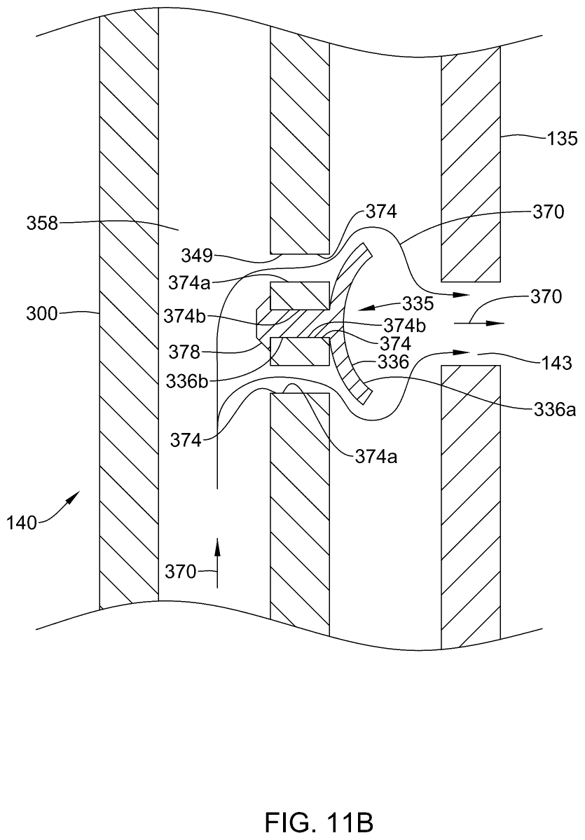

FIGS. 11A-15B schematically depict cross-section views of the seal assembly 335 positioned in, at, and/or proximate the third opening 349 to selectively control fluid between the third opening 349 of the shaft 300 of the gas/water valve 140 and the gas/air outlet 143 of the valve well 135. Although several configurations of the seal assembly are depicted in FIGS.>11A-15B, other suitable configurations thereof and/or relative to the shaft 300 and/or the valve well 135 are contemplated.

FIGS. 11A and 11B depict an illustrative configuration of the seal assembly 335 positioned between the third opening 349 of the shaft 300 and the gas/air outlet 143 to selectively block fluid between the third opening 349 and the gas/air outlet 143. The sub-openings 374 may be formed in a wall of the shaft 300 and may entirely or at least partially define the third opening 349. For example, and as depicted in FIGS. 11A and 11B, the third opening 349 may be defined by at least two gas/air sub-openings 374a (e.g., one or more gas/air sub-openings 374a may not be viewable in the cross-section view depicted) and one securing sub-opening 374b, but other suitable configurations are contemplated.

The seal assembly 335 may include the third seal 336 and the seal portion 336a thereof may be configured to overlap and seal the gas/air sub-openings 374a when the extension portion 336b is inserted into and/or through the securing sub-opening 374b to maintain the seal 336 at the third opening 349. For example, the seal portion 336a may have a diameter that extends beyond an outer-most portion of the gas/air sub-openings at the outer surface of the shaft 300. Although other configurations are contemplated, the extension portion 336b may be centered on the seal portion 336a. As depicted in FIGS. 11A and 11B, the extension portion 336b and the fixation component 378 thereof may be configured to fix a position of the extension portion 336b relative to the third opening 349 as discussed herein.

The third seal 336 may be coupled in or at the third opening 349 in any suitable manner. In one example, the extension portion 336b of the third seal 336 may be inserted into and/or through the securing sub-opening 374b such that the seal portion 336a is able to overlap and seal the gas/air sub-openings 374a when the seal is in the closed position and the fixation component 378 may engage an inner surface of the wall of the shaft 300 to fix the extension portion 336b relative to the third opening 349 and the shaft 300. Although the extension portion 336b may be inserted into the securing sub-opening 374b in one or more other manners, the extension portion 336b may be press-fit into the securing sub-opening 374b.

FIG. 11A depicts the gas/water valve 140 in the resting position in which gas/air is flowing out of a vent in the gas/water valve 140 (e.g., the vent opening 348, not shown in FIGS. 11A and 11B), as represented by arrows 362. When the gas/water valve 140 is in the resting position, the seal portion 336a of the third seal 336 may seal the gas/air sub-openings 374a and engage the outer surface of the shaft 300 while the extension portion 336b extends through the third opening 349 (e.g., through the securing sub-opening 374b) and engages the inner surface of the shaft 300, as depicted in FIG. 11A. In some examples, a negative pressure in the bore 358, other pressures proximate the seal portion 336a, and/or a bias force (e.g., from a resilient material, etc.) of the seal portion 336a may maintain the seal portion 336a in the closed position blocking gas/air from flowing through the gas/air sub-openings 374a at the third opening 349.

FIG. 11B depicts the gas/water valve 140 in the first position in which the vent is covered, which may cause a build-up of pressure in the bore 358 that applies a force to the seal portion 336a (e.g., a first or proximal side of the seal portion 336a) sufficient to adjust the third seal 336 from the closed position to the opened position. The seal portion 336a may be sufficiently flexible and/or resilient to bend or flex outward in response to pressure and/or forces acting on the first or proximal side or surface of the seal portion 336a proximate the gas/air sub-openings 374a, as depicted in FIG. 11B. When the seal portion 336a is in the opened position, gas/air may flow through the third opening 349, past the seal portion 336a, and through the gas/air outlet 143, as represented by the arrows 370. When the pressure in the bore 358 is equal to or lower than a pressure between the shaft 300 and the valve well 135, the seal 336 may return to the closed position.

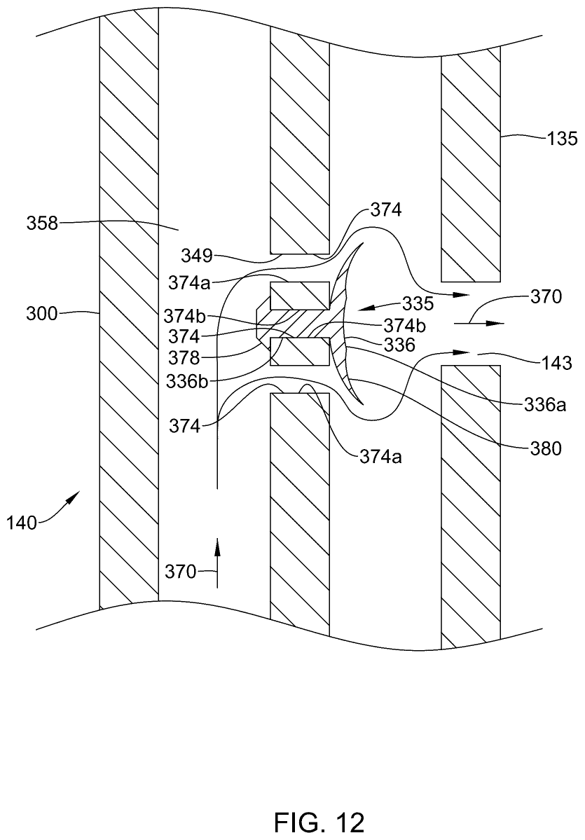

FIG. 12 depicts an illustrative configuration of the seal assembly 335 positioned between the third opening 349 of the shaft 300 and the gas/air outlet 143 to selectively block fluid between the third opening 349 and the gas/air outlet 143 similar to as depicted in FIGS. 11A and 11B, where the seal portion 336a of the third seal 336 may have a tapered portion 380 that tapers in the radially outward direction. In some examples, the tapered portion 380 may overlap the gas/air sub-openings 274a, which reduces the amount of pressure/force required to cause the seal portion to adjust from the closed position to the opened position as pressure in the bore 358 increases relative to exterior of the seal portion 336a. Other suitable tapered configurations of the seal portion 336a of the seal 336 are contemplated.

The seal portion 336a with the tapered portion 380 may be sufficiently flexible to bend or flex outward in response to pressure and/or forces acting on an inner surface of the seal portion 336a proximate the gas/air sub-openings 374a, as depicted in FIG. 12. When the seal portion 336a is in the opened position, gas/air may flow through the third opening 349, past the seal portion 336a and through the gas/air outlet 143, as represented by the arrows 370.

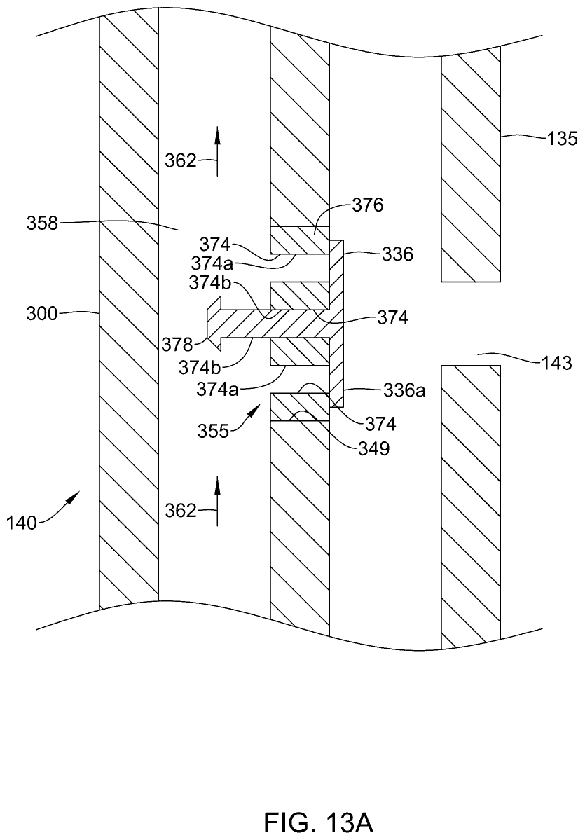

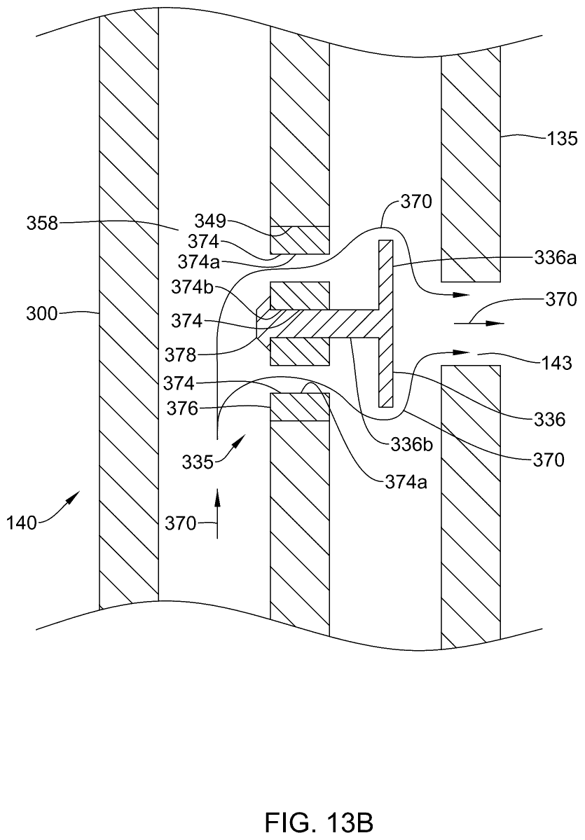

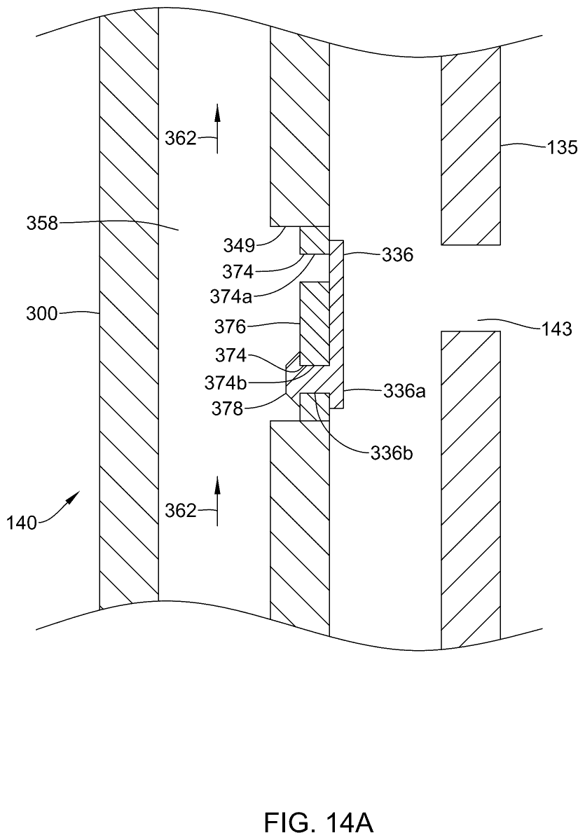

FIGS. 13A and 13B schematically depict an illustrative configuration of the seal assembly 335 positioned between the third opening 349 of the shaft 300 and the gas/air outlet 143 to selectively block fluid between the third opening 349 and the gas/air outlet 143. The seal assembly 335 may include the insert 376 and the third seal 336, where the sub-openings 374 may be formed in the insert 376. For example, and as depicted in FIGS. 13A and 13B, the third opening 349 may be defined by the wall of the shaft 300 and the insert 376 having at least two gas/air sub-openings 374a (e.g., one or more gas/air sub-openings 374a may not be viewable in the cross-section view depicted) and one securing sub-opening 374b may be inserted in the third opening 349. In some examples, the insert 376 may extend a width of the wall defining the third opening 349. Other suitable configurations of the insert 376 and/or the third opening 349 are contemplated.

The seal portion 336a of the third seal 336 may be configured to overlap and seal the gas/air sub-openings 374a when the extension portion 336b is inserted into and/or through the securing sub-opening 374b to maintain the seal 336 at the third opening 349. For example, the seal portion 336a may have a diameter that extends beyond an outer-most portion of the gas/air sub-openings at the outer surface of the shaft 300. Although other configurations are contemplated, the extension portion 336b may be centered on the seal portion 336a.

As depicted in FIGS. 13A and 13B, the extension portion 336b and the fixation component 378 thereof may be configured to maintain the seal 336 at the third opening 349, while allowing the seal to translate relative to a central axis of the shaft 300, the third opening 349, and/or the insert 376 (e.g., translate radially inward and outward). In one example, a length of the extension portion 336b between the fixation component 378 and the seal portion 336a may be longer than a depth of the securing sub-opening 374b such that the seal 336 may translate between a closed position and an opened position in response to changing pressure in the bore 358.

The gas/water valve 140 may be assembled by coupling the seal assembly 335 with the third opening 349 in any suitable manner. For example, the seal assembly 335 may be coupled with the third opening 349 by positioning the insert 376 in the third opening 349 and inserting the extension portion 336b of the third seal 336 into and/or through the securing sub-opening 374b such that the seal portion 336a is able to overlap and seal the gas/air sub-openings 374a when the seal 336 is in the closed position and the fixation component 378 may engage an inner surface of the insert 376 and maintain the third seal 336 at or proximate the third opening 349 when the seal 336 is in the opened position. Although the extension portion 336b may be inserted into the securing sub-opening 374b in one or more other manners, the extension portion 336b may be press-fit into the securing sub-opening 374b. The insert 376 may be inserted into the third opening 349 in any suitable manner discussed herein or otherwise.

FIG. 13A depicts the gas/water valve 140 in the resting position in which gas/air is flowing out of a vent in the gas/water valve 140 (e.g., the vent opening 348, not shown in FIGS. 13A and 13B), as represented by arrows 362. When the gas/water valve 140 is in the resting position, the seal portion 336a of the third seal 336 may seal the gas/air sub-openings 374a, the extension portion 336b may extend into and through the third opening 349, and the fixation component 378 may be spaced from the inner surface of the insert 376, as depicted in FIG. 13A. In some examples, a negative pressure in the bore 358 and/or other pressures proximate the seal portion 336a may maintain the seal portion 336a in the closed position blocking flow through the gas/air sub-openings 374a.

FIG. 13B depicts the gas/water valve 140 in the first position in which the vent is covered, which may cause a build-up of pressure in the bore 358 that applies a force to the seal portion 336a sufficient to adjust the third seal 336 from the closed position to the opened position. The seal portion 336a may be sufficiently rigid to cause the third seal 336 to translate outward (e.g., radially outward relative to a central axis of the shaft 300) in response to pressure and/or forces acting on the first or proximal surface of the seal portion 336a proximate the gas/air sub-openings 374a, as depicted in FIG. 13B. When the seal portion 336a is in the opened position, gas/air may flow through the third opening 349 via the gas/air sub-openings 374a, past the seal portion 336a and through the gas/air outlet 143, as represented by the arrows 370. When the pressure in the bore 358 is equal to or lower than a pressure between the shaft 300 and the valve well, the seal 336 may return to the closed position.

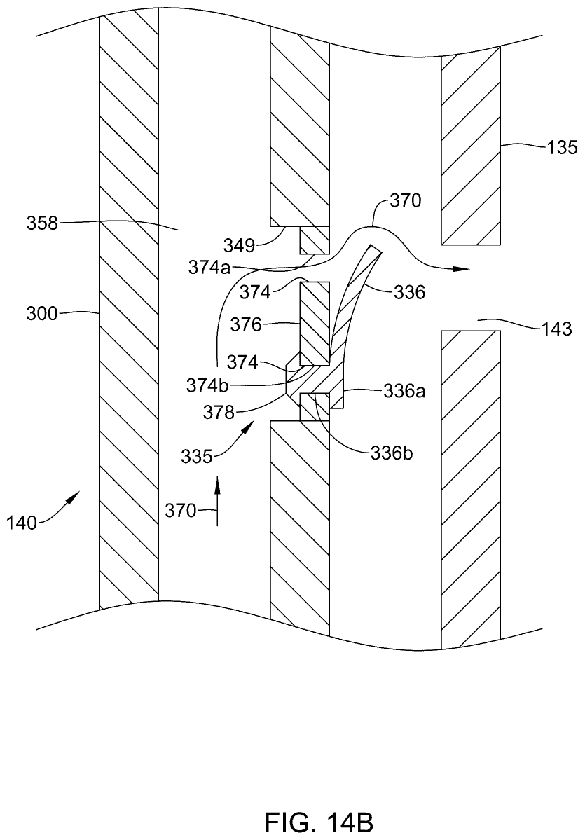

FIGS. 14A and 14B depict an illustrative configuration of the seal assembly 335 positioned between the third opening 349 of the shaft 300 and the gas/air outlet 143 to selectively block fluid between the third opening 349 and the gas/air outlet 143. The seal assembly 335 may include the insert 376 and the third seal 336, where the sub-openings 374 may be formed in the insert 376. For example, and as depicted in FIGS. 14A and 14B, the third opening 349 may be defined by the wall of the shaft 300 and the insert 376 having at least one gas/air sub-openings 374a (e.g., one or more gas/air sub-openings 374a may not be viewable in the cross-section view depicted) and one securing sub-opening 374b may be inserted in the opening. In some examples, the insert 376 may extend a width or depth that is less than a width or depth of the wall defining the third opening 349, as depicted in FIGS. 14A and 14B. Other suitable configurations of the insert 376 and/or the third opening 349 are contemplated.

The third seal 336 may be coupled in or at the third opening 349 in any suitable manner. In one example, the extension portion 336b of the third seal 336 may be inserted into and/or through the securing sub-opening 374b such that the seal portion 336a is able to overlap and seal the gas/air sub-openings 374a when the third seal 336 is in the closed position and the fixation component 378 is engaging an inner surface of the insert 376 to fix the extension portion 336b relative to the third opening 349, the shaft 300, and the insert 376, as depicted in FIGS. 14A and 14B. Although the extension portion 336b may be inserted into the securing sub-opening 374b in one or more other manners, the extension portion 336b may be press-fit into the securing sub-opening 374b.

The gas/water valve 140 may be assembled by coupling the seal assembly 335 with the third opening 349 in any suitable manner. For example, the seal assembly 335 may be coupled with the third opening 349 by positioning the insert 376 in the third opening 349 and inserting the extension portion 336b of the third seal 336 into and/or through the securing sub-opening 374b such that the seal portion 336a is able to overlap and seal the gas/air sub-openings 374a when the seal 336 is in the closed position and the fixation component 378 engages the inner surface of the insert 376. Although the extension portion 336b may be inserted into the securing sub-opening 374b in one or more other manners, the extension portion 336b may be press-fit into the securing sub-opening 374b. Further, the seal 336 may be coupled with the insert before, during, and/or after inserting the insert 376 into the third opening. The insert 376 may be inserted into the third opening 349 in any suitable manner discussed herein or otherwise.

FIG. 14A depicts the gas/water valve 140 in the resting position in which gas/air is flowing out of a vent in the gas/water valve 140 (e.g., the vent opening 348, not shown in FIGS. 14A and 14B), as represented by arrows 362. When the gas/water valve 140 is in the resting position, the seal portion 336a of the third seal 336 may seal the gas/air sub-opening(s) 374a, as depicted in FIG. 14A. In some examples, a negative pressure in the bore 358, other pressures proximate the seal portion 336a, and/or a bias force (e.g., from a resilient material, etc.) of the seal portion 336a may maintain the seal portion 336a in the closed position blocking gas/air from flowing through the gas/air sub-openings 374a.

FIG. 14B depicts the gas/water valve 140 in the first position in which the vent is covered, which may cause a build-up of pressure in the bore 358 that applies, via the gas/air sub-opening(s) 374a, a force to the first or proximal side of the seal portion 336a sufficient to adjust the third seal 336 from the closed position to the opened position. The seal portion 336a may be sufficiently flexible to bend or flex outward in response to pressure and/or forces acting on an inner surface of the seal portion 336a proximate the gas/air sub-opening(s) 374a, as depicted in FIG. 14B. When the seal portion 336a is in the opened position, gas/air may flow through the third opening 349, past the seal portion 336a, and through the gas/air outlet 143, as represented by the arrows 370. When the pressure in the bore 358 is equal to or lower than a pressure between the shaft 300 and the valve well 135, the seal 336 may return to the closed position.

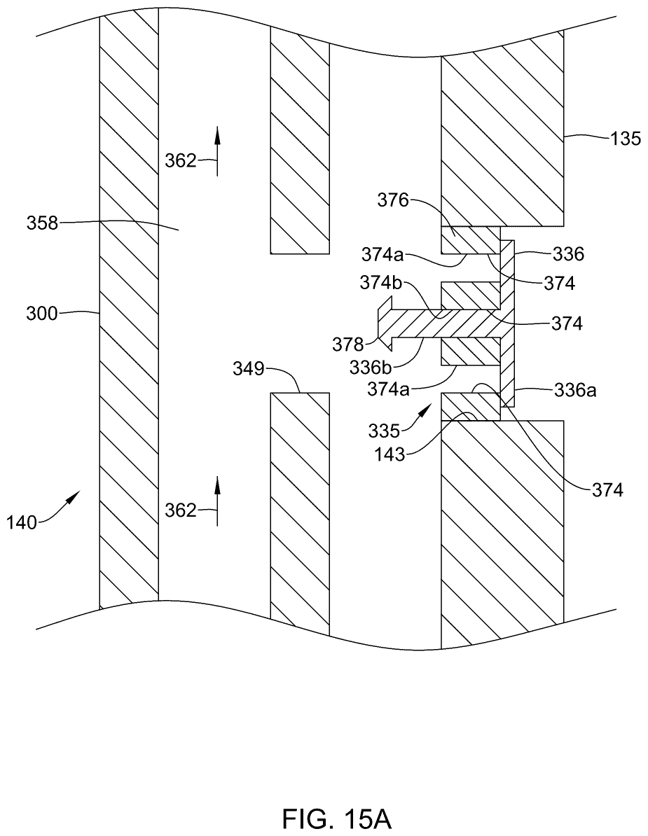

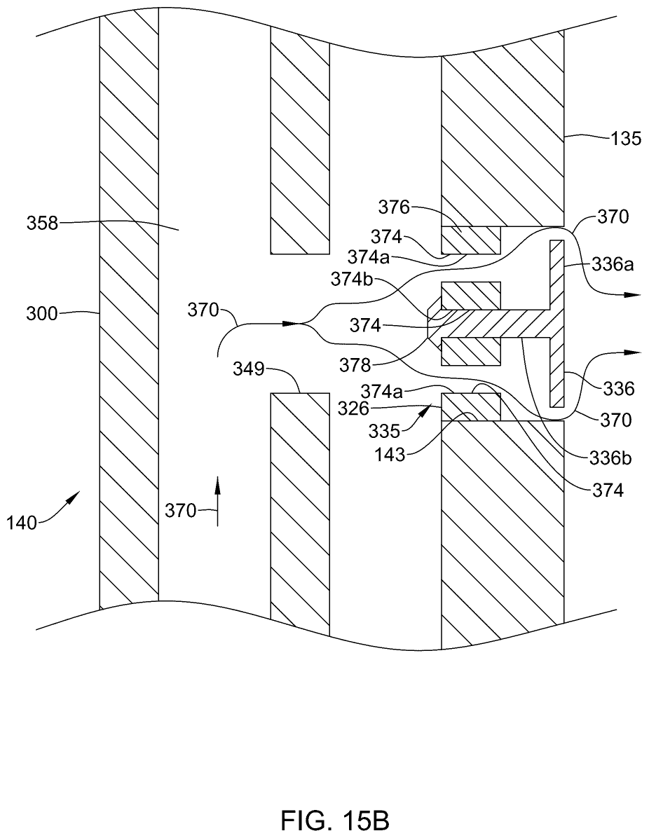

FIGS. 15A and 15B depict an illustrative configuration of the seal assembly 335 configured to selectively block fluid flow between the third opening 349 of the shaft 300 and the gas/air outlet 143. The seal assembly 335 may include the insert 376 and the third seal 336, where the sub-openings 374 may be formed in the insert 376. For example, and as depicted in FIGS. 15A and 15B, the third opening 349 may be defined by the wall of the shaft 300, the gas/air outlet 143 may be defined by a wall of the valve well, and the insert 376 having at least two gas/air sub-openings 374a (e.g., one or more gas/air sub-openings 374a may not be viewable in the cross-section view depicted) and one securing sub-opening 374b may be inserted in the gas/air outlet 143. In some examples, the insert 376 may extend a width of or less than the width of the wall defining the gas/air outlet 143. Other suitable configurations of the insert 376 and/or the gas/air outlet 143 are contemplated.

The seal portion 336a of the third seal 336 may be configured to overlap and seal the gas/air sub-openings 374a when the extension portion 336b is inserted into and/or through the securing sub-opening 374b to maintain the seal 336 at the third opening 349. For example, the seal portion 336a may have a diameter that extends beyond an outer-most portion of the gas/air sub-openings at the outer surface of the shaft 300. Although other configurations are contemplated, the extension portion 336b may be centered on the seal portion 336a.

As depicted in FIGS. 15A and 15B, the extension portion 336b and the fixation component 378 thereof may be configured to maintain the seal 336 at the gas/air outlet 143, while allowing the third seal 336 to translate relative the gas/air outlet 143 and/or the insert 376. In one example, the length of the extension portion 336b between the fixation component 378 and the seal portion 336a (e.g., the reduced diameter length) may be longer than a depth of the securing sub-opening 374b such that the seal 336 may translate between a closed position and an opened position in response to changing pressure in the bore 358.

The seal assembly 335 may be coupled with the gas/air outlet 143 in any suitable manner. For example, the seal assembly 335 may be coupled with the gas/air outlet 143 by positioning the insert 376 in the gas/air outlet 143 and inserting the extension portion 336b of the third seal 336 into and/or through the securing sub-opening 374b such that the seal portion 336a is able to overlap and seal the gas/air sub-openings 374a when the seal 336 is in the closed position and the fixation component 378 may engage a proximal side or surface of the insert 376 and maintain the third seal 336 at or proximate the third opening 349 and/or the gas/air outlet 143 when the seal 336 is in the opened position. Although the extension portion 336b may be inserted into the securing sub-opening 374b in one or more other manners, the extension portion 336b may be press-fit into the securing sub-opening 374b. The third seal 336 may be coupled with the insert 376 before, during, and/or after inserting the insert into the gas/air outlet 143. The insert 376 may be inserted into the gas/air outlet 143 in any suitable manner discussed herein for inserting the insert 376 into the third opening 349 or otherwise.

FIG. 15A depicts the gas/water valve 140 in the resting position in which gas/air is flowing out of a vent in the gas/water valve 140 (e.g., the vent opening 348, not shown in FIGS. 15A and 15B), as represented by arrows 362. When the gas/water valve 140 is in the resting position, the seal portion 336a of the third seal 336 may seal the gas/air sub-openings 374a and the fixation component 378 may be spaced from the proximal side or surface of the insert 376, as depicted in FIG. 15A. In some examples, a negative pressure in the bore 358 and/or the space between the shaft 300 and the valve well 135 proximate the third opening 349 and other pressures proximate the seal portion 336a may maintain the seal portion 336a in the closed position blocking flow through the gas/air sub-openings 374a.

FIG. 15B depicts the gas/water valve 140 in the first position in which the vent is covered, which may cause a build-up of pressure in the bore 358 and/or the space between the shaft 300 and the valve well 135 proximate the third opening 349 that applies a force to a proximal side of the seal portion 336a sufficient to adjust the third seal 336 from the closed position to the opened position. The seal portion 336a may be sufficiently rigid to cause the third seal 336 to translate outward in response to pressure and/or forces acting on an inner surface of the seal portion 336a proximate the gas/air sub-openings 374a, as depicted in FIG. 13B. When the seal portion 336a is in the opened position, gas/air may flow through the third opening 349, through the gas/air outlet 143 via the gas/air sub-openings 374a and past the seal portion 336a, as represented by the arrows 370. When the pressure in the bore 358 and/or the space between the shaft 300 and the valve well 135 is equal to or lower than a pressure distal of the seal portion 336a, the seal 336 may return to the closed position.

Various modifications and variations can be made to the disclosed device without departing from the scope of the disclosure. Other configurations of the disclosure will be apparent from consideration of the specification and practice of the invention disclosed herein. It is intended that the specification and examples be considered as illustrative only, with a true scope and spirit of the invention being indicated by the following claims.

All apparatuses and methods discussed herein are examples of apparatuses and/or methods implemented in accordance with one or more principles of this disclosure. These examples are not the only way to implement these principles but are merely examples. Thus, references to elements or structures or features in the drawings are references to examples of configurations of the disclosure, and do not limit the disclosure to the specific elements, structures, or features illustrated. Other examples of manners of implementing the disclosed principles will occur to a person of ordinary skill in the art upon reading this disclosure.

In the foregoing description and the following claims, the following will be appreciated. The phrases “at least one”, “one or more”, and “and/or”, as used herein, are open-ended expressions that are both conjunctive and disjunctive in operation. The term “a” or “an” entity, as used herein, refers to one or more of that entity. As such, the terms “a” (or “an”), “one or more” and “at least one” can be used interchangeably herein. All directional references (e.g., proximal, distal, upper, lower, upward, downward, left, right, lateral, longitudinal, front, back, top, bottom, above, below, vertical, horizontal, radial, axial, clockwise, counterclockwise, and/or the like) are only used for identification purposes to aid the reader's understanding of the present disclosure, and/or serve to distinguish regions of the associated elements from one another, and do not limit the associated element, particularly as to the position, orientation, or use of this disclosure. Connection references (e.g., attached, coupled, connected, and joined) are to be construed broadly and may include intermediate members between a collection of elements and relative movement between elements unless otherwise indicated. As such, connection references do not necessarily infer that two elements are directly connected and in fixed relation to each other. Identification references (e.g., primary, secondary, first, second, third, fourth, etc.) are not intended to connote importance or priority, but are used to distinguish one feature from another.

The foregoing discussion has been presented for purposes of illustration and description and is not intended to limit the disclosure to the form or forms disclosed herein. Various additions, modifications, and substitutions may be made to configurations disclosed herein without departing from the concept, spirit, and scope of the present disclosure. In particular, it will be clear to those skilled in the art that principles of the present disclosure may be embodied in other forms, structures, arrangements, proportions, and with other elements, materials, and components, without departing from the concept, spirit, or scope, or characteristics thereof. For example, various features of the disclosure are grouped together in one or more aspects or configurations for the purpose of streamlining the disclosure. However, various features of the certain aspects or configurations of the disclosure may be combined in alternate aspects or configurations. One skilled in the art will appreciate that the disclosure may be used with many modifications of structure, arrangement, proportions, materials, components, and otherwise, used in the practice of the disclosure, which are particularly adapted to specific environments and operative requirements without departing from the principles of the present disclosure. For example, elements shown as integrally formed may be constructed of multiple parts or elements shown as multiple parts may be integrally formed, the operation of elements may be reversed or otherwise varied, the size or dimensions of the elements may be varied, and features and components of various configurations may be selectively combined. The presently disclosed configurations are therefore to be considered in all respects as illustrative and not restrictive, the scope of the claimed invention being indicated by the appended claims, and not limited to the foregoing description.

The following claims are hereby incorporated into this Detailed Description by this reference, with each claim standing on its own as a separate configuration of the present disclosure. In the claims, the term “comprises/comprising” does not exclude the presence of other elements or steps. Furthermore, although individually listed, a plurality of means, elements or method steps may be implemented by, e.g., a single unit or processor. Additionally, although individual features may be included in different claims, these may possibly advantageously be combined, and the inclusion in different claims does not imply that a combination of features is not feasible and/or advantageous. In addition, singular references do not exclude a plurality. Reference signs in the claims are provided merely as a clarifying example and shall not be construed as limiting the scope of the claims in any way.

What is claimed is:

1. A gas/water valve for a medical device, the gas/water valve comprising:
a shaft having an outer surface, the shaft comprising:
a first opening;
a second opening;
a third opening located between the first opening and the second opening; and
a bore extending from the first opening to the second opening, the bore being in fluid communication with the third opening; and
a seal assembly positioned at the third opening and extending less than an entirety of a circumference around the shaft, the seal assembly configured to selectively seal the third opening.

2. The gas/water valve of claim 1, further comprising a first circumferential seal extending circumferentially around the shaft proximal of the third opening and a second circumferential seal extending circumferentially around the shaft distal of the third opening.

3. The gas/water valve of claim 1, wherein the seal assembly comprises an extension portion and a seal portion.

4. The gas/water valve of claim 3, wherein the extension portion is configured to extend into the third opening and engage an interior surface of the shaft and the seal portion is configured to engage the outer surface of the shaft and seal the third opening.

5. The gas/water valve of claim 3, wherein the seal portion is configured to translate radially relative to a central axis of the shaft.

6. The gas/water valve of claim 3, wherein the seal portion is resilient.

7. The gas/water valve of claim 3, wherein the seal portion is rigid.

8. The gas/water valve of claim 3, wherein the extension portion is centered on the seal portion.

9. The gas/water valve of claim 1, further comprising:
an insert positioned in the third opening, the insert comprising one or more securing openings and one or more gas/air openings.

10. The gas/water valve of claim 9, wherein the seal assembly comprises an extension portion configured to extend into a securing opening of the one or more securing openings and a seal portion configured to seal the one or more gas/air openings.

11. The gas/water valve of claim 10, wherein the insert has a depth proximate the securing opening and the extension portion has a reduced diameter length substantially equal to the depth.

12. The gas/water valve of claim 10, wherein the insert has a depth proximate the securing opening and the extension portion has a reduced diameter length greater than the depth, such that the extension portion is configured to translate radially relative to the insert as the seal portion adjusts between a closed position and an opened position.

13. The gas/water valve of claim 9, wherein the one or more gas/air openings comprise a first gas/air opening and a second gas/air opening.

14. A gas/water valve assembly for a medical device, the gas/water valve assembly comprising:
a valve well having a gas outlet;
a shaft positioned in the valve well, the shaft having an outer surface, the shaft comprising:
a first opening;
a second opening;
a third opening configured to align with the gas outlet; and
a bore extending from the first opening to the second opening, the bore being in fluid communication with the third opening; and
a seal assembly extending less than an entirety of a circumference around the shaft, the seal assembly configured to selectively block fluid from traveling between the third opening and the gas outlet.

15. The gas/water valve assembly of claim 14, wherein the shaft has a non-actuated position in the valve well and an actuated position in the valve well and the third opening is configured to align with the gas outlet when the shaft is in the non-actuated position.

16. The gas/water valve assembly of claim 14, further comprising:
an insert positioned in the third opening, the insert comprising one or more securing openings and one or more gas/air openings, and
wherein the seal assembly comprises an extension portion configured to extend into a securing opening of the one or more securing openings and a seal portion configured to seal the one or more gas/air openings.

17. The gas/water valve assembly of claim 14, further comprising:
an insert positioned in the gas outlet, the insert comprising one or more securing openings and one or more gas/air openings, and
wherein the seal assembly comprises an extension portion configured to extend into a securing opening of the one or more securing openings and a seal portion configured to seal the one or more gas/air openings.

18. The gas/water valve assembly of claim 14, further comprising:
a wiper seal positioned around the shaft between the first opening and the third opening, and
wherein the valve well comprises a gas inlet configured to align with the first opening and the wiper seal is configured to block fluid from traveling through the gas inlet to the gas outlet between an inner surface of the valve well and the outer surface of the shaft.

19. A method of assembling a gas/water valve, the method comprising:
positioning an insert in a third opening of a shaft comprising an outer surface, a first opening, a second opening, and a bore extending from the first opening to the second opening, wherein the third opening is positioned between the first opening and the second opening and is in fluid communication with the bore; and
inserting an extension portion of a seal assembly through a securing opening of the insert such that a seal portion of the seal assembly is configured to seal one or more gas/air openings of the insert.

20. The method of claim 19, wherein positioning the insert into the third opening of the shaft comprises over molding the insert at the third opening.
